(19) United States
(12) Patent Application Publication
(10) Pub. No.: US 2018/0042585 A1
(43) Pub. Date: Feb. 15, 2018

(54) SWEAT SENSING DEVICES WITH PRIORITIZED SWEAT DATA FROM A SUBSET OF SENSORS

(71) Applicant: University Of Cincinnati, Cincinnati, OH (US)

(72) Inventor: Jason C. Heikenfeld, Cincinnati, OH (US)

(21) Appl. No.: 15/551,306

(22) PCT Filed: Feb. 19, 2016
(86) PCT No.: PCT/US16/18635

§ 371 Date: Aug. 16, 2017

Related U.S. Application Data
(60) Provisional application No. 62/141,327, filed on Apr. 1, 2015.
(60) Provisional application No. 62/118,723, filed on Feb. 20, 2015.

(51) Int. Cl. A61B 10/00; A61B 5/00; G01N 33/487; A61B 5/145

(52) U.S. Cl. CPC A61B 10/0064; A61B 5/14517; A61B 5/4266; A61B 5/6833; G01N 33/48792

(57) ABSTRACT

A sweat sensor device for sensing at least one analyte includes a plurality of sensors for sensing said at least one analyte and for producing data. Data from a subset of said plurality of sensors is prioritized over data from sensors of said plurality of sensors which are outside said subset. A method of sweat sensing and prioritizing data in a device including a plurality of sensors includes determining a subset of sensors from said plurality of sensors by at least one sensing mechanism that measures a presence of or a property of sweat. The method further includes prioritizing data from a subset of the plurality of sensors over data from sensors of said plurality of sensors that are outside said subset.

### CROSS-REFERENCE TO RELATED APPLICATIONS

This application claims priority to U.S. Provisional Application Nos. 62/118,723 and 62/141,327, the disclosures of which are hereby incorporated by reference herein in their entirety.

### BACKGROUND OF THE INVENTION

Sweat sensing technologies have enormous potential for applications ranging from athletics, to neonatology, to pharmacological monitoring, to personal digital health, to name a few applications. Sweat contains many of the same biomarkers, chemicals, or solutes that are carried in blood and can provide significant information enabling one to diagnose ailments, health status, toxins, performance, and other physiological attributes even in advance of any physical sign. Furthermore, sweat itself, the action of sweating, and other parameters, attributes, solutes, or features on, near, or beneath the skin can be measured to further reveal physiological information.

If sweat has such significant potential as a sensing paradigm, then why has it not emerged beyond decades-old usage in infant chloride assays for Cystic Fibrosis or in illicit drug monitoring patches? Based on decades of sweat sensing literature, the majority of practitioners use the crude, slow, and inconvenient process of sweat stimulation, collection of a sample, transport of the sample to a lab, and then analysis of the sample by a bench-top machine and a trained expert. This process is so labor intensive, complicated, and costly that in most cases, one would just as well implement a blood draw since it is the gold standard for most forms of high performance biomarker sensing. Hence, sweat sensing has not emerged into its fullest opportunity and capability for biosensing, especially for continuous or repeated biosensing or monitoring. Furthermore, attempts at using sweat to sense “holy grails” such as glucose have not yet succeeded to produce viable commercial products, reducing the publicly perceived capability and opportunity space for sweat sensing.

Of all the other physiological fluids used for bio monitoring (e.g., blood, urine, saliva, tears), sweat has arguably the most variable sampling rate as its collection methods and variable rate of generation both induce large variances in the effective sampling rate. Sweat is also exposed to numerous contamination sources, which can distort the effective sampling rate or concentrations. The variable sampling rate creates a challenge in providing chronological assurance, especially so in continuous monitoring applications.

With improved sweat generation and sensing techniques, sweat sensing could become a compelling new paradigm as a biosensing platform.

### SUMMARY OF THE INVENTION

The present invention provides a sweat sensor device capable of reduced volume between the sensors and sweat glands. The present invention achieves this through reduced sensor areas and volumes. An embodiment of the present invention includes a sweat sensor device for sensing at least one analyte including a plurality of sensors for sensing said at least one analyte and for producing data. Data from a subset of said plurality of sensors is prioritized over data from sensors of said plurality of sensors which are outside said subset.

A further embodiment of the present invention includes a method of sweat sensing and prioritizing data in a device including a plurality of sensors. The method includes determining a subset of sensors from said plurality of sensors by at least one sensing mechanism that measures a presence of or a property of sweat and prioritizing data from a subset of the plurality of sensors over data from sensors of said plurality of sensors that are outside said subset.

### BRIEF DESCRIPTION OF THE DRAWINGS

The objects and advantages of the present invention will be further appreciated in light of the following detailed descriptions and drawings in which:

FIG. 1A is a cross-sectional view of a portion of a device according to an embodiment of the present invention before sweat has been generated.

FIG. 1B is a cross-sectional view of the portion of the device of FIG. 1A after sweat has been generated and sensed.

FIG. 2 is a cross-sectional view of a portion of a device according to another embodiment of the present invention after sweat has been generated and sensed.

FIG. 3 is a cross-sectional view of a portion of a device according to another embodiment of the present invention after sweat has been generated and sensed.

FIG. 4 is a cross-sectional view of a portion of a device according to another embodiment of the present invention after sweat has been generated and collected.

FIG. 5 is a cross-sectional view of a portion of a device according to another embodiment of the present invention after sweat has been generated and sensed.

FIG. 6 is a cross-sectional view of a portion of a device according to another embodiment of the present invention before sweat has been generated and sensed.

### DEFINITIONS

As used herein, “continuous monitoring” means the capability of a device to provide at least one measurement of sweat determined by a continuous or multiple collection and sensing of that measurement or to provide a plurality of measurements of sweat over time.

As used herein, “chronological assurance” is an assurance of the sampling rate for measurement(s) of sweat or solutes in sweat in terms of the rate at which measurements can be made of new sweat or its new solutes as originating from the body. Chronological assurance may also include a determination of the effect of potential contamination with previously generated sweat, previously generated solutes, other fluid, or other measurement contamination sources for the measurement(s).

As used herein, “determined” may encompass more specific meanings including but not limited to: something that is predetermined before use of a device; something that is determined during use of a device; something that could be a combination of determinations made before and during use of a device.

As used herein, “sweat sampling rate” is the effective rate at which new sweat or sweat solutes, originating from the sweat gland or from skin or tissue, reaches a sensor which measures a property of sweat or its solutes. Sweat sampling rate, in some cases, can be far more complex than just sweat generation rate. Sweat sampling rate directly determines or is a contributing factor in determining the chronological assurance. Times and rates are inversely proportional (rates having at least partial units of 1/seconds), therefore a short or small time required to refill a sweat volume can also be said to have a fast or high sweat sampling rate. The inverse of sweat sampling rate (1/s) could also be interpreted as a “sweat sampling interval” (s). Sweat sampling rates or intervals are not necessarily regular, discrete, periodic, discontinuous, or subject to other limitations. Like chronological assurance, sweat sampling rate may also include a determination of the effect of potential contamination with previously generated sweat, previously generated solutes, other fluid, or other measurement contamination sources for the measurement(s). Sweat sampling rate can also be in whole or in part determined from solute generation, transport, advective transport of fluid, diffusion transport of solutes, or other factors that will impact the rate at which new sweat or sweat solutes reach a sensor and/or are altered by older sweat or solutes or other contamination sources.

As used herein, “sweat stimulation” is the direct or indirect causing of sweat generation by any external stimulus, the external stimulus being applied for the purpose of stimulating sweat. One example of sweat stimulation is the administration of a sweat stimulant such as pilocarpine. Going for a jog, which stimulates sweat, is only sweat stimulation if the subject jogging is jogging for the purpose of stimulating sweat.

As used herein, “sweat generation rate” is the rate at which sweat is generated by the sweat glands themselves. Sweat generation rate is typically measured by the flow rate from each gland in nL/min/gland. In some cases, the measurement is then multiplied by the number of sweat glands from which the sweat is being sampled.

As used herein, “active control of sweat sampling rate” is where an external stimulus is applied to skin or the body to change or control the sweat generation rate and therefore the sweat sampling rate. This may also be more directly referred to as “active control of sweat generation rate.”

As used herein, “measured” can imply an exact or precise quantitative measurement and can include broader meanings such as, for example, measuring a relative amount of change of something. Measured can also imply a binary measurement, such as ‘yes’ or ‘no’ type measurements.

As used herein, a “determined sweat generation rate” is one that is determined during use of a sweat measuring device.

As used herein, a “predetermined sweat generation rate” is one that is determined from a method other than during use of a sweat measuring device that uses predetermined sweat generation rate to provide chronological assurance.

As used herein, “sweat volume” is the fluidic volume in a space that can be defined multiple ways. Sweat volume may be the volume that exists between a sensor and the point of generation of sweat or a solute moving into or out of sweat from the body or from other sources. Sweat volume can include the volume that can be occupied by sweat between: the sampling site on the skin and a sensor on the skin where the sensor has no intervening layers, materials, or components between it and the skin; or the sampling site on the skin and a sensor on the skin where there are one or more layers, materials, or components between the sensor and the sampling site on the skin.

As used herein, a “predetermined sweat volume” is one that is determined before use of a sweat measuring device.

As used herein, a “determined sweat volume” is one that is determined during use of a sweat measuring device.

As used herein, “solute generation rate” is simply the rate at which solutes move from the body or other sources into sweat. “Solute sampling rate” includes the rate at which these solutes reach one or more sensors.

As used herein, “microfluidic components” are channels in polymer, textiles, paper, or other components known in the art of microfluidics for guiding movement of a fluid or at least partial containment of a fluid.

As used herein, “advective transport” is a transport mechanism of a substance or conserved property by a fluid due to the fluid's bulk motion.

As used herein, “diffusion” is the net movement of a substance from a region of high concentration to a region of low concentration. This is also referred to as the movement of a substance down a concentration gradient.

As used herein, “convection” is the concerted, collective movement of groups or aggregates of molecules within fluids and rheids, either through advection or through diffusion or a combination of both.

As used herein, “predetermined solute transport” is solute transport other than advective transport that is determined before use of a sweat measuring device.

As used herein, “measured solute transport” is solute transport other than advective transport that is determined during use of a sweat measuring device.

As used herein, a “volume-reduced pathway” is a sweat volume that has been reduced by addition of a material, device, layer, or other body-foreign substance, which therefore decreases the sweat sampling interval for a given sweat generation rate. This term can also be used interchangeably in some cases with a “reduced sweat pathway”, which is a pathway between eccrine sweat glands and sensors that is reduced in terms of volume or in terms of surfaces wetted by sweat along the pathway. Volume reduced pathways or reduced sweat pathways include those created by sealing the surface of skin, because skin can absorb or exchange water and solutes in sweat which could increase the sweat sampling interval and/or cause contamination, which can also alter the accuracy or duration of the sweat sampling interval. Volume reduced pathways may also include the volume required by the sensor itself to contact sweat.

As used herein, “volume reducing component” means any component which reduces the sweat volume. In some cases, the volume reducing component is more than just a volume reducing material, because a volume reducing material by itself may not allow proper device function (e.g. for example the volume reducing material would need to be isolated from a sensor for which the volume reducing material could damage or degrade, and therefore the volume reducing component may comprise the volume reducing material and at least one additional material or layer to isolate volume reducing material from said sensors).

As used herein, a “horizontally-confining component” is a component that does not allow fluid to substantially spread horizontally along the skin surface.

### DETAILED DESCRIPTION OF THE INVENTION

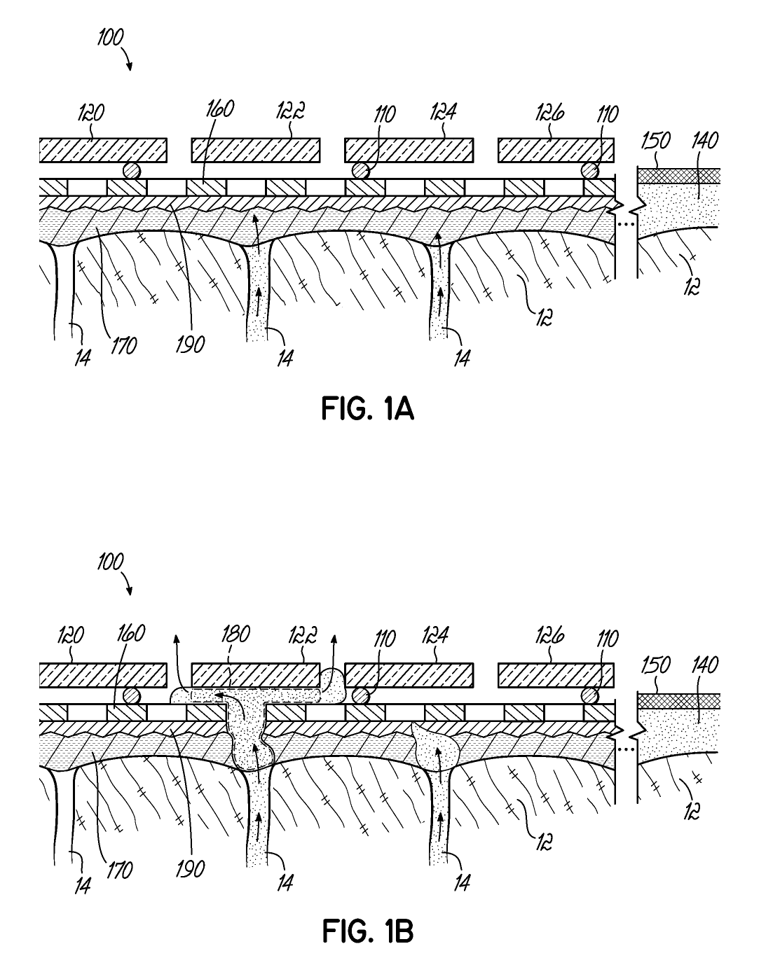

Embodiments of the present invention are generally directed to sweat sensor devices including a subset of sensors that have a greater influence on the overall sensor data than sensors outside of that subset. With reference to FIG. 1A, in one embodiment, a device 100 optionally includes a membrane component 160, a volume reducing component 170, a sweat dissolvable material 190, and a spacer material 110. Device 100 further includes the sensors 120, 122, 124, 126, which are described further below. Skin 12 is shown as including one or more sweat gland ducts 14. The membrane component 160 may be, for example, a track etch membrane. The volume reducing component 170 could be petroleum jelly or a silicone oil or cosmetic oil that is insoluble in sweat. In one embodiment, the volume reducing component 170 may be electrically insulating. By way of example, if the volume reducing component 170 and membrane component 160 were adjacent each other, the volume reducing component 170 could clog the pores in the membrane component 160 and the pressure of sweat generation may then be inadequate to allow sweat to push through the membrane component 160. Such difficulty can be resolved by the addition of the sweat dissolvable material 190.

By way of example, the sweat dissolvable material 190 could be constructed of materials such as sucrose, table salt, polyvinyl alcohol, polyethylene oxide, or any other suitable material. The sweat dissolvable material 190 may be, for example, fabricated onto the membrane component 160 by using a track-etch membrane, which is roller coated and microreplicated with the sweat dissolvable material 190. The spacer material 110 may separate the set of sensors 120, 122, 124, 126 from the membrane component 160. In one embodiment, the spacer material 110 may support a 10 μm microfluidic gap between the set of sensors 120, 122, 124, 126 and the membrane component 160. The spacer material 110 may be, for example, microspheres of glass or polymer, a layer of silica gel, or a layer of cellulose (not shown). With reference to FIG. 1B, the device 100 is shown after sweating has commenced by stimulation or by natural means. The resulting sweat volume 180 is shown in dashed line and comprises the entire or a majority of the area under the sensor 122. Sweat volume and techniques for the reduction thereof are further described in International Application No. PCT/US2015/0032893, the disclosure of which is hereby incorporated by reference herein in its entirety.

With further reference to FIGS. 1A and 1B, the sensors 120, 122, 124, 126 measure one or more solutes in sweat or the presence or flow rate of sweat. The set of sensors 120, 122, 124, 126 are all similar by having the same sensing mechanisms and targeted analytes. In one embodiment, the sensors 120, 122, 124, 126 may all be glucose sensors and the targeted analyte may be glucose. While the set is specifically shown as including four sensors 120, 122, 124, 126, it will be appreciated that the set may have more than two and less than four sensors or may have more than four sensors. The number and size of sensors in the set are configured such that, on average, each set will cover at least one active sweat gland duct 14. As a result, the sweat volume that is related to one or a few sweat gland ducts 14 may be related to a subset of the set of sensors. For example, the sweat passing through the sweat volume 180 (shown in dashed line) will be primarily sensed by the sweat sensor 122.

Using a set of sensors may allow for a reduction in the sampling interval time as compared to a similar device including one relatively larger sensor. As shown in the calculations for Example 1 below, for the case of one relatively larger sensor, the sampling interval may be very large (e.g., tens of minutes or more). The same can be true for the device 100 shown in FIG. 1 if the sensors 120, 122, 124, 126 were replaced with a single, larger sensor (not shown). The sweat volume in the case of the device 100 with the single, larger sensor would be dominated by the space between the sensor and the membrane component 160. A single, smaller sensor would reduce the sampling interval time, but a single, smaller sensor may not, on average, cover an active sweat gland duct. In other words, a single smaller sensor (e.g., sensor 120) has a lower chance of being placed over an active eccrine sweat gland duct. Thus, a set of sensors (e.g., sensors 120, 122, 124, 126) allows for a reduction in the sample interval time while retaining the likelihood that an active sweat gland duct will be accounted for by a sensor. If, for example, the set of sensors uses sensors that are 0.25 mm in diameter, the sweat volume between each sensor and the membrane component 160 is reduced by 100λ. However, a 0.25 mm diameter sensor would have an area of only 0.05 mm², and, for the case of 100 active sweat glands/cm², there would only be a 5% chance that a single small sensor would cover an active sweat gland. Therefore, in one embodiment, sixty of the relatively small sensors could be placed such that, on average, three of the sensors would be placed above an active sweat gland.

In an aspect of the present invention, the data that comes from sensors directly above or closest to an active sweat gland duct 14 is prioritized over data from sensors that are not close to an active sweat gland duct 14. For example, sensors that show a sweat signal first or a lower electrical impedance with sweat or skin could be determined to be those that should be prioritized for the reading of sweat data. As a result, this subset of sensors will have greater influence on the overall sensor data than similar sensors outside the subset.

The determination of which sensors are to be in the subset of sensors that will be prioritized could be achieved in multiple ways, including but not limited to: (1) determining which sensors receive sweat first; (2) determining which sensors measure the changes to concentrations in analytes in the shortest time period; and (3) implementing a local fluid flow rate measurement such as flow meters used in the microfluidics field. It should be recognized that electronics and computing, microcontrollers, circuits, smartphones with wireless connection to the device, and other methods can be utilized to, or to help in, determining which sensors are to be prioritized. In various embodiments of the present invention, the sensors and resulting data that is not prioritized could be: recorded, but not presented to the user; not even recorded or saved; or flagged as being due to older sweat and presented differently to the user. Further, the data that is prioritized could be, for example: shown or reported to the user while the unprioritized data is not; analyzed while the unprioritized data is not; flagged as being more reliable than the unprioritized data; or could be giving a higher weighting in an average data response calculated from all of the sensors.

Several examples now described further illustrate aspects of the present invention. If electrical impedance (capacitance and/or resistance) were measured at each sensor 120, 122, 124, 126 in the state of the device 100 shown in FIG. 1A, the electrical resistance measured at each sensor would be near infinite and the electrical capacitance would be low because the sensors would only be exposed to gas (i.e., air) and the sensors would be separated from skin by electrically insulating oil. In the state of the device 100 shown in FIG. 1B, sweat contacts the sensor 122, such that it now senses the electrical resistance of the skin 12 and of an eccrine duct 14 filled with sweat, which is much lower in resistance and capacitance than the skin 12 alone, as is known to those skilled in the art. As a result, in one embodiment, sensor 122 would receive a higher priority for the sensor data it produces or could even be the only sensor from which sensor data would be utilized. In this specific example, only 25% of the sweat sensors (i.e., sensors 120, 122, 124, 126) would be utilized in sensing and reporting at least one analyte in sweat, such as glucose. In another embodiment, as seen in FIG. 1B, sensor 124 is likely to receive sweat before sensors 120 and 126, and therefore sensors 122 and 124 could be prioritized together, such that 50% of the sensors are prioritized and the other sensors are disregarded for analyte sensing. The number of sensors in the subset of sensors that are prioritized compared to the total number of sensors will vary based on the device design, the sweat sensing conditions, and the particular application.

As described previously, the sweat sampling rate will also be faster when the data from the subset of sensors is prioritized. For example, in the case of a 2.5 mm vs. 0.25 mm sensor described above, there was a 100× reduction in sweat volume which could result in a similar increase in sweat sampling rate. Therefore, another way to prioritize the sensors would be based on those which provide the fastest responses to changes in analyte concentrations, which depends at least in part on the sweat sampling rate. For example, where sensors 120, 122, 124, 126 are ion-selective or conductivity sensors for sodium, the concentration of which changes rapidly with sweat rate, sensors 122, 124 could show changes in sodium concentrations that occur in only minutes, whereas sensors 120, 126 could show changes in sodium concentrations over periods of tens of minutes. As a result, sensors 122, 124 would be prioritized and data from sensors 120, 126 unprioritized.

Embodiments of the present invention may include features, surfactants, or other aspects that promote wetting of sweat to the sweat dissolvable material 190 or wetting of sweat through volume reducing component 170 to membrane component 160. All such techniques are herein referred to as sweat-wetting promoting features. In the embodiment illustrated in FIG. 1A, the sweat dissolvable material 190 may be a microreplicated film with spikes to promote wetting and dissolution by sweat. Without a non-planar rough or spiky surface, even with sweat pressure, a sweat impermeable film of volume reducing component 170 may exist longer than desired between sweat and the sweat dissolvable material 190. Some embodiments of the present invention may nevertheless include a sweat dissolvable material 190 having a non-spiky surface.

Also shown in FIGS. 1A and 1B is a sweat stimulating component consisting of a driving electrode 150 and a sweat stimulant 140. As discussed above, the volume reducing component 170 may clog or seal the skin 12. Consequently, in a configuration where sweat stimulation occurs in approximately the same location as the volume reducing component 170, the volume reducing component 170 would prevent or substantially hinder the initiation of sweating by iontophoresis. Separating the area of sweat stimulation from the volume reducing component 170 solves this problem. As shown in FIG. 1A, the driving electrode 150 and sweat stimulant 140 are separate from the volume reducing component 170. The sweat stimulant 140 may be, for example, a gel containing a sweat stimulant such as pilocarpine, methacholine, or carbachol. By way of example, the driving electrode 150 and sweat stimulant 140 may be located within 1 cm of the location where sweat will be sensed. In the illustrated embodiment, the sweat stimulation is achieved by sudo-motor axon reflex sweating, such that the device 100 can be applied to initially dry and non-sweating skin 12. Sweat may be stimulated by, for example, iontophoresis without the need for fluidic contact between the sweat stimulant 140 and the skin 12 beneath the sensors 120, 122, 124, 126. Sudo-motor axon reflex sweating and mechanisms for achieving the same are further described in U.S. Provisional Application No. 62/115,851 the disclosure of which is hereby incorporated by reference herein in its entirety. Other solutions are possible and included within the present invention, and this specific example is shown only to show that the present invention may include sweat stimulation. Other mechanisms of sweat stimulation may be included in embodiments of the present invention. For example, a stimulation device (not shown) may be used to stimulate sweat on skin 12, then the stimulation device may be removed from that stimulated area, and then a device according to the present invention may be placed onto that same area for sweat sampling and sensing purposes.

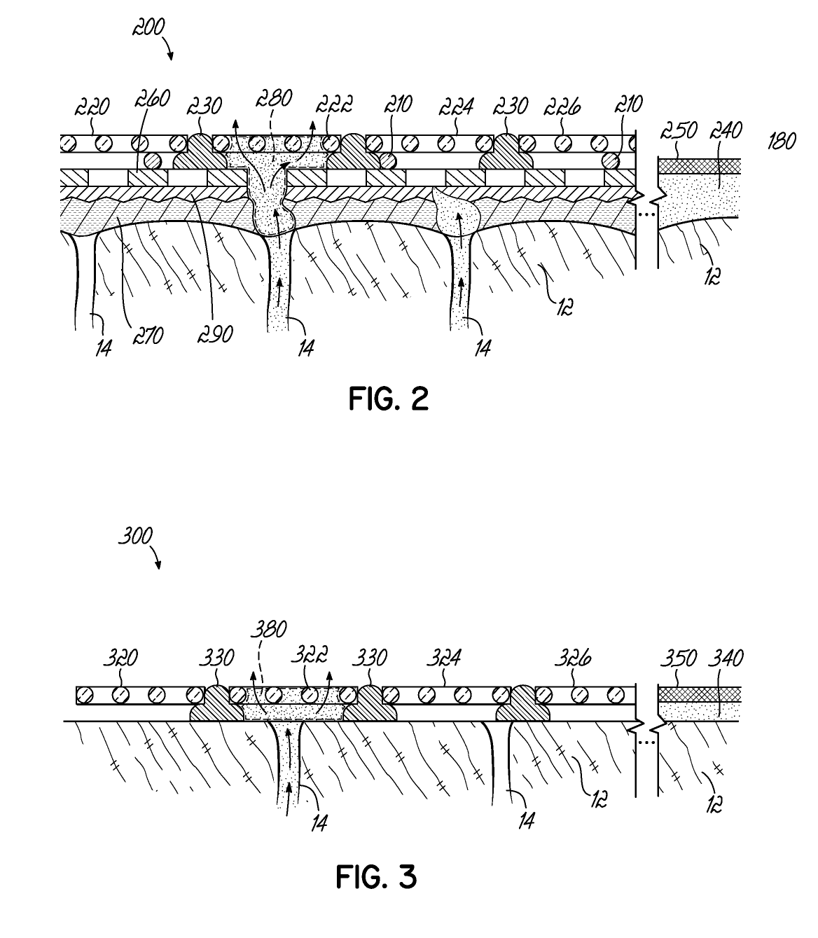

With reference to FIG. 2, in one embodiment, the device 200 includes a volume reducing component 270, a membrane component 260, a sweat stimulating gel 240, a driving electrode 250, a spacer material 210, and an adhesive 230. The adhesive 230 at least partially fluidically isolates the sensors 220, 222, 224, 226 from one another. The adhesive could be an acrylate adhesive epoxy that is UV cured, for example. The sweat volume 280 is shown in dashed line between the sensor 222 and the skin 12. Furthermore, the sensors, such as sensor 222, can be partially or highly porous such that sweat has an escape path from beneath the sensors. Sensors 220, 222, 224, 226 could also be pressure-permeated or may include a pressure-permeated component (not shown) such that only sensors above active sweat glands would receive sweat. For example, sensors could be ion-selective sensors that are further coated on the upper surface (i.e., the surface opposite the skin-facing surface) with a thin hydrophobic monolayer of fluoropolymer, such as Teflon. This fluidic isolation can reduce the effective sweat volume beneath each sensor by reducing or eliminating the amount of adjacent old sweat that could mix with newly generated sweat underneath the sensor. This fluidic isolation can also improve the ability to identify which sensors should be prioritized for sensing, because this fluidic isolation reduces the likelihood of sensors not above active sweat ducts being wetted with sweat. For example, only sensors receiving sweat with pressure from below would be wetted with sweat, and other sensors would not be wetted even if sweat were to touch their upper surface (e.g., because above those sensors there would be no pressure to push sweat through those sensors or because the upper surface of those sensors is sealed).

With reference to FIG. 3, in one embodiment, the device 300 includes a set of sensors 320, 322, 324, 326, an adhesive 330, a driving electrode 350, and a sweat stimulating gel 340. In this embodiment, the adhesive 330 is used to create a reduced sweat volume 380 directly on skin 12. For example, adhesive 330 can be a pressure sensitive adhesive, which promotes robust and compliant contact with the skin 12. Unlike the adhesive 230 shown in FIG. 2, where a variety of adhesives may be used, in FIG. 3, the adhesive 330 touches skin and should be a skin compatible adhesive, such as those used commercially in medical adhesives and tapes.

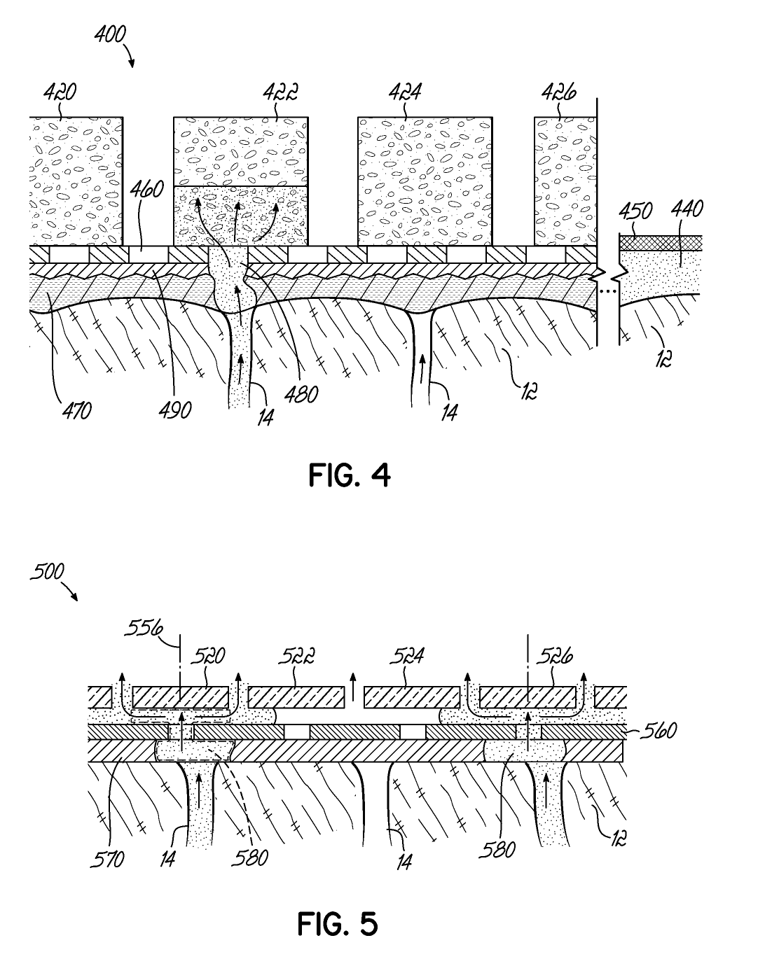

With reference to FIG. 4, in one embodiment comprising a sweat collection device 400, the device 400 includes a volume reducing component 470, a membrane component 460, a sweat stimulating gel 440, driving electrode 450, and collection members 420, 422, 424, 426. Like sweat sensing, highly pure and rapid sweat collection can benefit from minimizing the amount of contamination of sweat from skin (i.e., by minimizing the sweat dead volumes that are filled that result in sweat not collected) and by isolating sweat from individual sweat glands. As disclosed herein, the device 400 includes a sweat volume that is limited to the collection member 422. Collection member 422 could be, for example, an array of glass capillaries (not shown to scale) that has a slightly hydrophobic coating at its surface facing toward the skin 12 such that wicking of sweat only occurs into collection member 422 and not horizontally between the capillary and the membrane component 460. Collection member 422 could also be glued or sealed against the membrane component 460 to achieve the same effect. The collection member 422 could then allow extraction of sweat which is then taken to an external sensor or measurement equipment to determine one or more properties of sweat. In one embodiment, the collection members 420, 422, 424, 426 could comprise a single collection member, although a small increase in sweat dead volume and mixing of samples from multiple sweat glands would then be possible.

It should be recognized that a collection member may be used in combination with elements of other devices disclosed herein. For example, each sensor illustrated in FIG. 2 could have its own collection element to wick away old sweat. Because the collection elements would be isolated, sweat would not reach the other sensors that are not located above an active sweat gland. As a result, those sensors would not provide a sensed signal, further ensuring the proper sensing of sweat.

With reference to FIG. 5, in one embodiment, the device 500 includes a volume reducing component 570 and a membrane 560. As indicated by the dashed line 556, the sensor 520 has a sensor-centered volume reduced pathway 580 for sweat coming from skin 12 and sweat duct 14, which decreases the sampling interval. The membrane 560 is a sweat impermeable material that centers the flow of sweat along the axis as indicated by the dashed line 556. Having a sensor-centered volume reduced pathway is advantageous because, if sweat flow is not centered, then a portion of the sensor will have non-uniform and slower flow. In particular, a flow of sweat near at least a first region would be slower than the flow at other regions under the sensor. This slow sweat flow will cause old sweat to be measured along with new sweat and effectively increase the sampling interval. Thus, for a sensor placed on the skin 12, even if the sensor size is relatively small, or if techniques disclosed herein reduce sweat volumes between the sweat ducts and the sensor, the sensor could receive a flow of sweat that is not centered under the sensing surface, and therefore would not see an optimal sweat sampling rate. For a sensor having a circular sensor area, flow into the center of the sensor would be optimal in terms of minimizing mixing of simultaneous readings of old sweat and new sweat. While the term ‘centered’ may not exactly apply to sensors with non-circular sensor geometries, aspects of the present invention include a sensor configuration that receives sweat in a manner that results in an optimum or near optimum sampling interval. In other words, a sensor-centered volume reduced pathway includes a predetermined pathway across sensors for sweat, which decreases the sampling interval. It should be recognized that device 500 and similar embodiments may be constructed using a variety of methods. By way of example, device 500 may be constructed by aligned lamination of films, by photolithography, or by other means.

In one advantageous aspect of the present invention, a sensor may be porous to sweat. Including a sensor porous to sweat may reduce the time needed for new sweat to flush old sweat away from sensors. Additionally, if the center of the sensor is porous, then the flow of sweat would be centered or uniform through the sensor (hence ‘centered flow’ can also be meant to include ‘uniform flow’). As described above, sweat volume can be reduced by using centered flow (e.g., using the configuration of device 500). However, the sweat volume can be further reduced and faster sweat sampling rates enabled by prioritizing the data from a subset of smaller sized sensors as taught in previous embodiments. In one embodiment, sensors 520, 522, 524, 526 could all be sensors for cortisol and each have a diameter of 100 μm. Those sensors receiving sweat first, such as 520 and 526, could be prioritized over other sensors for measurement and reporting of cortisol in sweat.

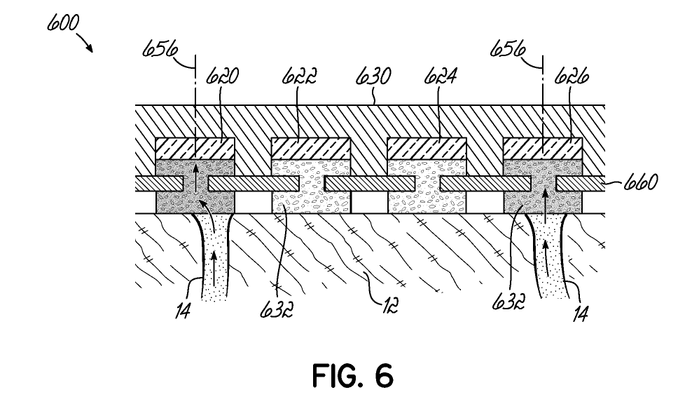

With reference to FIG. 6, in one embodiment, the device 600 includes a sweat impermeable material 660, sensors 620, 622, 624, 626, and sweat wicking components 632, 630. Sweat wicking component 632 could be, for example, cellulose or a hydrogel having a 10-20 μm thickness between the sweat impermeable material 660 and the skin 12 and having a similar thickness between the sweat impermeable material 660 and the sensor 622. The sweat wicking component 630 could be a sponge, gel, textile, fibrous material, or other material which further wicks sweat as received from the sweat wicking component 632. In an embodiment where the sweat impermeable material (e.g., a membrane or mesh) 660 were adequately hydrophilic, the sweat wicking component 632 could be removed and sweat could wick by capillary action between the sweat impermeable material 660 and both the skin 12 and the sensor 622.

The devices shown in FIGS. 1A through 6 can be easily manufactured. For example, 0.25 mm electrodes are easily fabricated even on flexible substrates, with much smaller sizes possible. Electrode arrays can all be functionalized together with a general coating or film covering all, as needed, requiring a comparable number of steps as are needed for making a single sensor. Multiplexors or other electronics can also be located near the sensors, if needed, to help reduce complex wiring or to increase signal quality. Therefore, each sensor described above in various embodiments of the present invention could also represent a plurality of sensors, and in some cases even including local electronics for control of the sensor or buffering/amplifying the sensor signal. For example, such sensors could be manufactured by silicon manufacturing techniques, with one sensor component as represented in the drawings comprising arrays of sensors that are each microscale in size.

Embodiments of the present invention may be useful for a variety of sweat sensing applications. In one instance, low sweat rates enabled by embodiments of the present invention can also allow sensing of some solutes that otherwise might be difficult. For example, a large sweat rate can cause the sweat gland itself to generate significant lactate, and hopelessly complicate the correlation of sweat lactate to blood lactate. Because of the reduced sweat rate required by embodiments of the present invention, blood lactate that partitions into sweat ducts or glands may be allowed to be dominant over lactate generated by the sweat gland. Therefore, embodiments of the present invention enable improved measurement of lactate through sweat ducts or glands. Embodiments of the present invention could also help in sensing of cytokines, which partition into sweat very slowly and likely require slow sweat rates for high quality sensing. Embodiments of the present invention can also help by reducing the amount of stimulation needed for a given sampling interval or chronological resolution by reducing the sweat volume needed by the sensors, which in turn reduces the sweat generation rate needed to refresh that sweat volume. Similarly, the present invention could also reduce the time for a new concentration of biomarkers to move from blood into sweat and onto the sensors, therefore providing a sweat measurement that is closer to a real time assessment in the biomarker in blood.

The following examples are provided to help illustrate the present invention, and are not comprehensive or limiting in any manner.

### Example 1

For simplicity, we can assume for purposes of illustration that the minimum sweat generation rate on average is about 0.1 nL/min/gland and the maximum sweat generation rate is about 5 nL/min/gland, which is about a 50× difference between the two. Consider a single sensor which must cover at least 3 active sweat glands on an area of skin with 100 active glands/cm². This would require a minimum sensor area of 3 mm² (0.03 cm²). Assume the gap between skin and the sensor is on average 30 μm. The volume beneath the sensor and the skin is therefore 30E-4 cm×0.03 cm²=9E-5 mL or 90 nL. At sweat generation rates of 5, 1, and 0.1 nL/min/gland, it would require 6, 30, or 300 minutes, respectively, to fill this volume. However, this is a calculation for a best case scenario, because the flow is somewhere between homogeneous (sweat glands everywhere) and centered (one single source of sweat in the center of the sensor). As a result, the time for filling of a fresh (new) sample of sweat could be about 6× greater than in the simplified calculation (i.e., 36, 180, 1800 minutes, respectively).

### Example 2

Again assume 100 active glands/cm², and that at least 3 sweat glands and sensors are to overlap, as taught using an embodiment of the present invention similar to that described for FIG. 6. This would require about 3 mm² of capture area. Assume the sensors placed over the capture area are 500 μm in diameter or about 0.2 mm² (0.002 cm²) in area each. In 3 mm² of capture area, 15 such sensors could be placed in, for example, a hexagonal packing pattern. The volume beneath each sensor would be 30E-4 cm×0.002 cm²=6E-6 mL (6 nL). At sweat generation rates of 5, 1, and 0.1 nL/min/gland, it would require 1.2, 6, or 60 minutes, respectively, to fill this volume. However, this is a calculation for a best case scenario, because the flow is somewhere between homogeneous and centered. As a result, the time for filling of a fresh (new) sample of sweat could be about 6× greater than in the simplified calculation (i.e., 7.2, 36, 360 minutes, respectively). In any of these cases, the sweat sampling rate at which new sweat replaces old sweat is 5× faster than that of Example 1. The sweat sampling rate could be even faster, using any technique taught here or otherwise possible to reduce the sweat volume between sensors and skin.

### Example 3

Using Example 2 as a reference, the sensors could be made smaller using silicon manufacturing at 50 μm in diameter. This would be 100× smaller area, and 1500 sensors per 3 mm². At a sweat generation rate of 0.1 nL/min/gland, it would require about 36 seconds to fill this volume.

Embodiments of the present invention apply at least to any type of sweat sensor device that measures sweat, sweat generation rate, sweat chronological assurance, its solutes, solutes that transfer into sweat from skin, a property of or things on the surface of skin, or properties or things beneath the skin. Embodiments of the present invention applies to sweat sensing devices which can take on forms including patches, bands, straps, portions of clothing, wearables, or any suitable mechanism that reliably brings sweat stimulating, sweat collecting, and/or sweat sensing technology into intimate proximity with sweat as it is generated. Devices according to embodiments of the present invention could be held near the skin by adhesives or by other mechanisms that hold the device secure against the skin, such as a strap or embedding in a helmet. Further, sweat sensor devices may be in wired communication or wireless communication with a reader device. For example, a reader device may be a smart phone or portable electronic device. For purposes of brevity and to focus on the inventive aspects, the embodiments described above are shown diagrammatically in the figures, and it should be recognized that certain components (e.g., a battery) may be included depending on the application, although not explicitly described. For example, a counter electrode may be included in a device when iontophoresis is the chosen sweat stimulation method.

While all of the present disclosure has been illustrated by a description of various embodiments, and while these embodiments have been described in considerable detail, it is not the intention of the Applicant to restrict or in any way limit the scope of the appended claims to such detail. Additional advantages and modifications will readily appear to those skilled in the art. The invention in its broader aspects is therefore not limited to the specific details, representative apparatus and method, and illustrative examples shown and described. Accordingly, departures may be made from such details without departing from the spirit or scope of the Applicant's general inventive concept.

What is claimed is:

1. A sweat sensor device for sensing at least one analyte comprising:
a plurality of sensors for sensing said at least one analyte and for producing data;
wherein data from a subset of said plurality of sensors is prioritized over data from sensors of said plurality of sensors which are outside said subset.

2. The device of claim 1, further comprising:
at least one volume-reducing component providing a volume-reduced pathway for sweat between said plurality of sensors and sweat glands.

3. The device of claim 2, wherein each of said sensors includes an associated sweat volume that is isolated to each of said sensors.

4. The device of claim 2, wherein said at least one volume-reducing component comprises a sweat wicking material.

5. The device of claim 1, further comprising:
a plurality of collection elements,
wherein each of said sensors corresponds with one of said collection elements.

6. The device of claim 1, wherein each of said sensors has a volume reduced sweat pathway that is centered on the sensor.

7. The device of claim 1, wherein said subset of said plurality of sensors includes those sensors that receive sweat first.

8. The device of claim 1, wherein said subset of said plurality of sensors includes those sensors that most quickly measure a change in a concentration of said at least one analyte.

9. The device of claim 1, further comprising:
at least one flow sensor associated with each of said sensors for measuring a flow of sweat fluid,
wherein said subset of said plurality of sensors includes those sensors that have the fastest flow of sweat fluid as measured by said at least one associated flow sensor.

10. A sweat sampling device comprising:
a plurality of collection elements for collecting at least one analyte;
wherein a subset of said plurality of collection elements sample sweat and those sensors of said plurality of sensors which are outside the subset do not sample sweat.

11. The device of claim 10, further comprising:
a volume-reducing component providing a volume-reduced pathway for sweat between said collection elements and sweat glands.

12. A method of sweat sensing and prioritizing data in a device comprising a plurality of sensors, the method comprising:
determining a subset of sensors from said plurality of sensors by at least one sensing mechanism that measures a presence of or a property of sweat;
prioritizing data from a subset of the plurality of sensors over data from sensors of said plurality of sensors that are outside the subset.

13. The method of claim 12, wherein determining the subset of sensors includes determining which sensors from said plurality of sensors receive sweat first.

14. The method of claim 12, wherein determining the subset of sensors includes determining which sensors from said plurality of sensors receive a volume of new sweat in a shorter time period than those sensors that are outside the subset.

15. The method of claim 12, wherein determining the subset of sensors includes determining which sensors from said plurality of sensors measure a change in a concentration of at least one analyte in a shortest time period.

16. The method of claim 12, wherein prioritizing the data from said subset of sensors includes analyzing the data from said subset of sensors and not analyzing data from the sensors that are outside the subset.

17. The method of claim 12, wherein prioritizing the data from said subset of sensors includes labeling data from the sensors that are outside the subset.
